(19) United States
(12) Plant Patent Application Publication
(10) Pub. No.: US 2013/0326755 P1
(43) Pub. Date: Dec. 5, 2013

(54) Rosa hybrid variety denomination 'Poulcas036'

(50) ;

(71) Applicant: Mogens Nyegaard Olesen, Fredensborg (DK)

(72) Inventor: Mogens Nyegaard Olesen, Fredensborg (DK)

(21) Appl. No.: 13/507,022

(22) Filed: May 31, 2012

(51) Int. Cl. A01H 5/00

(52) U.S. Cl. PLT/142

(57) ABSTRACT

A new garden rose plant of the Compact Floribunda class which has abundant, yellow and red bi-color flowers and attractive foliage. This new and distinct variety has shown to be uniform and stable in the resulting generations from asexual propagation.

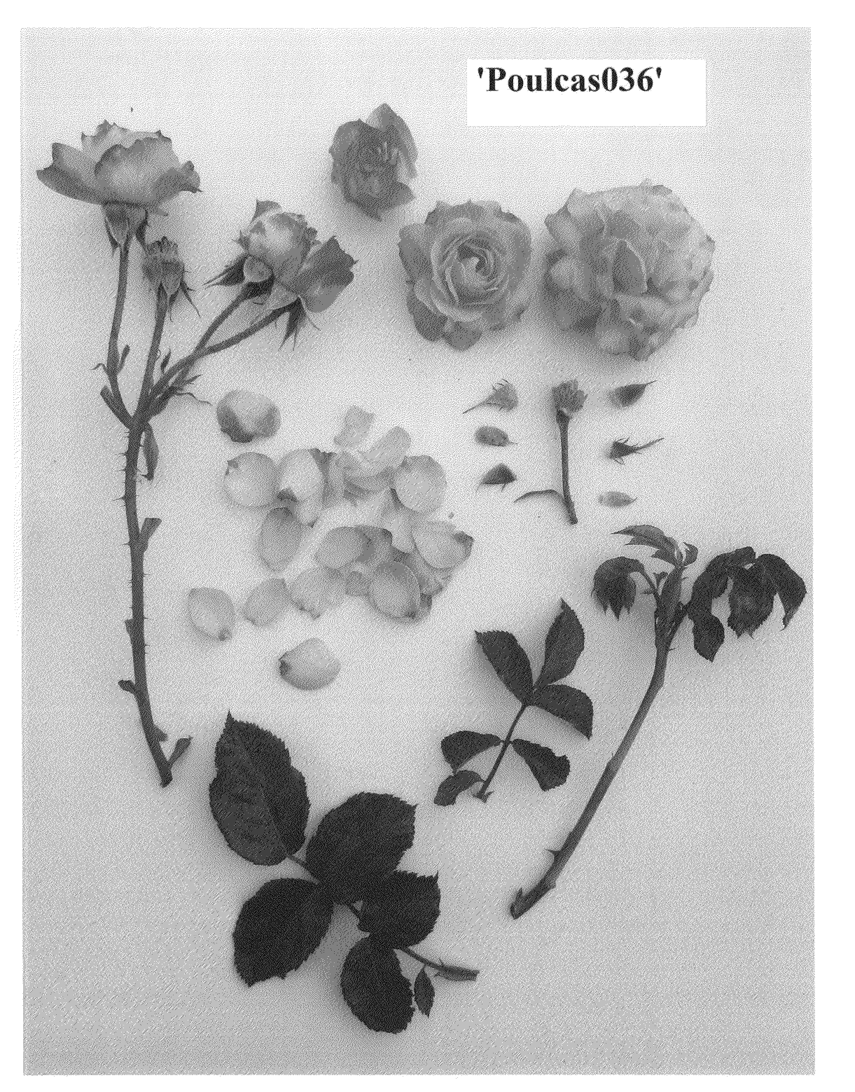

### BOTANICAL DESIGNATION

Rosa hybrid

### VARIETY DENOMINATION

‘Poulcas036’

### SUMMARY OF THE INVENTION

The present invention constitutes a new and distinct variety of garden rose plant which originated from a controlled crossing between the female seed parent, an unnamed seedling, and the male pollen parent, also an unnamed seedling.

The two parents were crossed during the summer of 2003 and the resulting seeds were planted in a controlled environment in Fredensborg, Denmark. The new variety, named ‘Poulcas036’, originated as a single seedling from the stated cross.

The new variety may be distinguished from its male pollen parent and female seed parent primarily by flower coloration and growth habit.

The objective of the hybridization of this rose variety was to create a new and distinct variety for garden use with unique qualities, such as:

1. Uniform and abundant yellow and red bi-color flowers;

2. Vigorous, but compact growth when propagated both as a budded rose and on its own roots;

3. Exceptional disease resistance.

This combination of qualities is not present in previously available commercial cultivars of this type, known to the inventor, and distinguish ‘Poulcas036’ from all other varieties of which we are aware.

As part of the rose development program, Mogens N. Olesen germinated the seeds from the aforementioned hybridization during winter of 2003 and conducted evaluations on the resulting seedlings in a controlled environment in Fredensborg, Denmark. ‘Poulcas036’ was selected in the spring of 2004 by the inventor as a single plant from the progeny of the aforementioned hybridization.

Asexual reproduction of ‘Poulcas036’ by traditional budding and rooted cuttings was first done by Mogens N. Olesen in the nursery in Fredensborg, Denmark in July, 2004. This initial and other subsequent asexual propagations conducted in controlled environments have demonstrated that the characteristics of ‘Poulcas036’ are true to type and are transmitted from one generation to the next.

### DESCRIPTION OF THE DRAWING

The accompanying color illustration shows as true as is reasonably possible to obtain in color photographs of this type, the typical characteristics of the buds, flowers, leaves, and stems, of ‘Poulcas036’. Specifically illustrated in the drawing are flowers at various stages of development, flower in parts, leaves, and stems.

### DETAILED DESCRIPTION OF THE VARIETY

The following is a description of ‘Poulcas036’, as observed in its growth in in a field nursery in Marion County, Oreg. Observed plants are 3 years of age, and were grown on their own roots. Color references are made using The Royal Horticultural Society (London, England) Colour Chart, 2001, except where common terms of color are used.

For a comparison, several physical characteristics of the rose variety ‘Jacrite’, U.S. Plant Pat. No. 8,361 are compared to ‘Poulcas036’ in Chart 1.

CHART 1

| | ‘Poulcas036’ | ‘Jacrite’ |
|---|---|---|
| Petal Count | 35 | 25 |
| Flower | 50 to 60 mm | 4.5 to 5 inches |
| Diameter | | (110 to 120 mm) |
| Flower Color | Upper surface: The base of | The upper side of the petals is |
| | the petal is Yellow Group | Yellow Group 6C on the inner |
| | 12A, middle zone is Yellow- | petals. On the reverse side of |
| | Orange Group 14C, margins | the petals, the inner petals are |
| | are Red Group 47B | Yellow Group 6C while the |
| | and 47C. Lower surface: | outer are Red Group 40D, |
| | Mid and basal zones are | except at the petal base where |
| | Yellow-Orange Group 14C. | they are Yellow Group 6C. |
| | Margins are Red Group 47B. | The base of the petals is |
| | | Yellow Group 6C. The major |
| | | color on the upper side is |
| | | Yellow Group 6C. |

Flower and flower bud:
   Blooming habit.—Continuous.
   Flower bud.—Size: Upon opening, 23 mm in length from base of receptacle to end of bud. Bud diameter is 12 mm. Bud form: Ovoid. Bud color: As sepals divide petals are Yellow Group 13C, Red Group 45A, and Red Group 46C. Sepal inner surface: Color: Yellow-Green Group 147C with intonations of Greyed-Red Group 178B. Surface: Smooth with strong pubescence. Sepal outer surface: Color: Yellow-Green Group 144A with weak intonations of Greyed-Red Group 178B. Texture: Somewhat rough with many stipitate glands. Sepal Shape: Apex: Cirrhose. Base: Flat at union with receptacle. Sepal Margin: Margins have moderate foliaceous appendages on three of the five sepals. Sepal size: 23 mm long by 6 mm wide. Receptacle: Texture: Smooth. Shape: Urn shaped. Size: 7 mm in height by 7 mm wide. Color: Yellow-Green Group 144A with intonations of Greyed-Red Group 178B. Pedicel: Surface: Somewhat rough with small prickles. Length: 25 to 35 mm. Diameter: 3 mm on average. Color: Yellow-Green Group 144A with moderate anthocyanic pigments the color of Greyed-Red Group 178A. Strength: Strong. Peduncle: Length: 20 to 25 cm. Diameter: 3 to 4 cm. Color: Yellow-Green Group 144A with moderate anthocyanic pigments the color of Greyed-Red Group 178A. Texture: Somewhat rough with many small prickles.
   Flower bud development.—Flower buds are borne in clusters of 3 to 5 flower buds per stem, resembling a corymb.
   Flower bloom.—Fragrance: Light floral scent. Duration: The blooms have a duration on the plant of approximately 7 to 10 days. Petals fall cleanly away from plant after flowers have fully matured. Size: Flower diameter is 50 to 60 mm when open. Flower depth is 25 mm. Flower shape: General shape is a high centered, semi double, with a high pointed center which is tightly closed. Shape of flower, side view: Upon opening the upper portion is flat. The lower portion is a flattened convex.
   Petalage.—Under normal conditions, flowers have 35 petals total, 3 to 5 of which are petaloids.
   Petal color.—Upon opening, outer petals Upper surface: The base of the petal is Yellow Group 12A, middle zone is Yellow-Orange Group 14C, margins are Red Group 47B and 47C. Lower surface: Mid and basal zones are Yellow-Orange Group 14C. Margins are Red Group 47B. Upon opening, inner petals: Upper surface: Yellow Group 12A with shaded intonations of Orange-Red Group 26C at the margins. Margins have other intonations of Red Group 47B. Lower surface: Yellow Group 13C with marginal spots of Red Group 43A. After opening, outer and inner petals: Upper surface: Yellow Group 12A at the petal base. Yellow-Orange Group 14C at the middle zone, with intonations of Orange-Red Group 26C. Margins are splashed with Red Group 47B. Lower surface: Yellow-Orange Group 14C. The margins are Yellow-Orange Group 18D splashed with Red Group 47B.
   General tonality.—Open flowers are Yellow Group 11A and 13D with intonations of Red Group 52A and 52D.
   Petals.—Petal Reflex: Weak. Margin: Entire and uniform with a point at the center. Weak undulations of margin observed. Shape: Generally narrow elliptic. Apex shape: Cuspidate. Base shape: Acute. Size: 25 mm (l)×28 mm (w). Texture: Smooth. Thickness: Average.
   Petaloids.—Quantity: 3 to 5. Shape: Obovate, with a rounded apex and acute base. Color: Yellow Group 9B with intonations of Yellow-Orange Group 23C on upper surface. Lower surface is Yellow Group 12C and Yellow-Orange Group 22B. Size: 15 mm (l)×11 mm (w).
   Reproductive organs.—Pollen: None observed. Anthers: Size: 2 mm in length. Color: Yellow Group 11B. Quantity: 65 on average. Filaments: Color: Yellow-Orange Group 14A. Length: 6 mm. Pistils: Length: 6 mm. Quantity: 35 on average. Stigmas: Color: Greyed-Yellow Group 160B. Styles: Color: Greyed-Yellow Group 160B. Location of stigmas: Level in location relative to the length of the filaments and the height of the anthers. Hips: None Observed.
Plant:
   Plant growth.—Upright, bushy. Plants are 40 cm in height, and 40 cm wide.
   Stems.—Color: Juvenile growth: Yellow-Green Group 144B with anthocyanin the color of Greyed-Orange Group 176B. Mature growth: Yellow-Green Group 144A. Length: On average, canes are 30 cm from the base of the plant to the flowering portion. Diameter: 4 mm. Internodes: On mature canes, there is an average distance of 30 mm between nodes. Surface Texture: Young wood: Smooth. Older wood: Rough.
   Long prickles.—Incidence: 3 prickles per 10 cm of stem. Size: Average length of prickles on mature stems is 6 mm. Shape: Upper portion is linear. Lower portion is deep concave. Color: Juvenile prickles: Greyed-Red Group 181 A. Mature prickles: Greyed-Yellow Group 160A.
   Plant foliage.—Compound leaf: 140 mm (l)×100 (w). Quantity: 3 leaves per 10 cm of stem on average. Leaf bearing angle to the stem: 60 degrees. Color of juvenile foliage: Upper side: Yellow-Green Group 144A. Lower side: Yellow-Green Group 144B. Anthocyanin: Greyed-Orange Group 175A generalized. Color of mature foliage: Upper side: Yellow-Green Group 146A. Lower side: Yellow-Green Group 146B.
   Plant leaves and leaflets.—Stipules: Size: 20 mm in length. Quantity: 2 per compound leaf. Shape: Linear, slightly broad based with outward extending apecies. Margins: Finely serrated with few stipitate glands. Color: Yellow-Green Group 144A. Petiole: Length: 20 mm. Diameter: 3 mm. Upper surface: Color: Yellow-Green Group 144A with anthocyanin the color of Greyed-Orange Group 176A. Lower surface: Color: Yellow-Green Group 144B. Observations: Numerous stipitate glands and prickles observed. Rachis: Length: 65 mm long. Upper surface: Color: Yellow-Green Group 144A. Lower surface: Color: Yellow-Green Group 144B. Observations: Numerous stipitate glands and prickles observed. Leaflet: Quantity: Normal number of leaflets leaves in middle of the stem is 5 leaflets. Margins: Serrated. Size: Average size of the terminal leaflet on normal leaves is 60 mm in length by 35 mm wide. Shape: Generally elliptical. Base: Rounded. Apex: Mucronate. Texture: Somewhat rough. Thickness: Average. Arrangement: Odd pinnate. Venation: Reticulate.
   Glossiness.—Moderately glossy.
   Disease resistance.—Above average resistance to powdery and downy mildew, rust, black spot, and Botrytis under normal growing conditions.
   Cold hardiness.—The variety is tolerant to USDA Cold Hardiness Zone 6.
   Heat tolerance.—The variety has been found to be suitable for climate conditions found in the American Horticulture Society heat zone 7.

1. A new and distinct variety of rose plant of the Compact Floribunda rose class named ‘Poulcas036’, substantially as illustrated and described herein, due to its abundant yellow and red bi-color flowers, disease resistance, and extended period of bloom.
